(19) United States
(12) Patent Application Publication
(10) Pub. No.: US 2006/0241455 A1
(43) Pub. Date: Oct. 26, 2006

(54) Automatically adjusted presets for an ultrasound machine

(75) Inventor: Ann Shvarts, San Jose, CA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Medical Solutions USA, Inc.

(21) Appl. No.: 11/102,520

(22) Filed: Apr. 8, 2005

(51) Int. Cl. A61B 8/00

(52) U.S. Cl. 600/447

(57) ABSTRACT

There is a need for a better way of collecting information on adjusting and determining presets in ultrasound machines. The system and method according to various embodiments of the present invention involve automatic collection of data of ultrasound scan settings frequently used and optimized by the operator for various types of scan scenarios. With the automatically collected data, the ultrasound machine operator is given the opportunity to accept modifications to the default presets or to prompt creation of new presets. Additionally, the automatically collected information is reported back to the manufacturing company and used for determining what default presets should be programmed in new machines. The report back is done in real time through wireless connection, network connection or both. Alternatively, the report back is done through use of a storage device or computer readable memory medium in which ultrasound scan settings are saved for later review.

### FIELD OF THE INVENTION

The present invention relates to analysis of medical imaging presets and in particular to monitoring, determination, and/or adjustment of presets in ultrasound machines.

### BACKGROUND

Ultrasound or pulse-echo sonography is a diagnostic imaging technique which uses high-frequency acoustic energy to examine tissue structures. Ultrasound scans are made by ensonifying a target area with high-frequency acoustic pulses (ultrasound waves) and receiving reflections of these pulses at the probe (transducer) as they reflect from an object or the boundary of a region encountered in that area. The received reflections of the pulses (echo waves) are processed electronically and converted into a visual image of the ensonified region, which is usually displayed on a monitor or stored for later analysis. Ultrasound waves reflect from boundaries between tissues of differing acoustic impedance such as organs and surrounding fluid or between regions of different tissue density. Modern ultrasound provides dynamic real-time imaging capability of tissue structure, tissue motion, and various tissue parameters, either directly or by implication of observation of secondary features such as ultrasound contrast agents.

Ultrasound has many modalities which are used for examining various tissues. These modalities include B-mode imaging, continuous wave spectral (CW) Doppler, pulse wave spectral (PW) Doppler and color flow Doppler. Using these modalities, cardiac ultrasound, or cardiac echocardiography and peripheral vascular ultrasound, are widely used as effective diagnostic tools for heart and blood vessel diseases. The 2-dimensional echocardiogram reveals the hemodynamic parameters of blood flowing through arteries and veins, e.g., speed and direction of blood flow within the heart helpful in evaluating the heart valve function. Peripheral vascular ultrasound is used to obtain images and measure the velocity of blood flow in blood vessels such as arteries and veins in the neck, abdomen, legs and arms.

Ultrasound tests such as echocardiograms involve a complex composite of parameters, some of which are controllable by the operator (sonographers or physician) and some of which are determined by the physical properties of the patient's body. Because of the dynamic range and variance of the reflected signals, and the complexity of the interactions within the body, it is desirable to set guidelines for adequately covering the various scenarios. Accordingly, ultrasound machines come equipped with parameter control settings which are predefined by the manufacturer and are useful for initial imaging settings. These predefined settings for imaging parameters are commonly referred to as presets.

For example, the Nyquist criterion dictates the sample rate (2×PRF), that is the highest sampling rate for a particular PRF. Pulse repetition frequency (PRF) is pulse rate, namely, the number of pulses transmitted in a second. PRF determines, for instance, the maximum measurable velocity of fluid flowing in a blood vessel. To avoid range ambiguity in the measurement, one form of aliasing, pulse echoes from structures must return to the transducer before a next pulse is transmitted. To avoid frequency aliasing, the PRF needs to be kept high enough to prevent velocity aliasing. Combined, these operate to limit the maximum detectable velocity when measuring deep structures, and more complicated techniques must be employed to measure deep depth high velocity flow. Thus, control knob adjustments for setting the PRF allow the user to take into consideration the interaction between PRF and the depth of structures to be examined, particularly in large patients.

Another parameter affecting the Doppler measurement governs the wall filter, which reject signals based on frequency. Wall filters eliminate reflections from stationary or relatively slow moving objects that produce low frequency shifts by eliminating signals at the lower frequency shifts (wall filters maintain echoes from the fast moving objects, e.g., blood flow). Additional scan parameters may include transmit and receive frequency, transmit and receive apodization, receive aperture growth, pulse shaping such as pulse length, polarity, coding, chirp rise rate and frequency span characteristics, dynamic range, dynamic focus, persistence, color map, gain, line density, number of lines, focal zones, focal depth, and image span. Some parameters are mode specific, like angle correction for spectral Doppler or sweep rate for M-mode. Yet other parameters enable or control manufacturer specific modes such as Siemens Sequoia™ ultrasound system's TEQ™ technology, or Siemens Antares™ ultrasound system's Clarify™ technology. Yet other parameters control the display or various third party vendor's peripherals. There is a large number of parameters controlling various aspects of the ultrasound system which are either set or are potentially modifiable through the system's user interface.

Parameters for controlling user interface and display, including how keys are defined, how images are displayed and how image data is stored may involve different settings for different types of ultrasound exams. These and other parameters are preset for various imaging scenarios by the ultrasound machine manufacturer and then manually adjusted by the operator to suit their preferences, office procedures or special cases.

Thus, when an ultrasound machine is delivered it is pre-programmed with a number of default presets optimized for specific types of ultrasound tests and patient profiles. Many ultrasound machines are further equipped with a preset store functionality with which, subsequent to manually adjusting the parameters, an operator can name the new combination of presets and add them to the original manufacturer's list of default presets. Then, for patient exams with previously determined requirements, the operator can select the saved presets from the list.

However, disrupting a patient exam to use the preset store functionality is not practical. Committing preferred preset combinations to memory for post exam preset adjustment and store is also impractical and inconvenient. Likewise, experienced users often adjust parameters to create and store preferred combinations, but less experienced users may not necessarily find such combinations and readily recognize them for what they are. In addition, there is currently no way to inform an operator, who may repeatedly, but unknowingly, adjust the ultrasound machine to the same presets combination, that these settings may be saved for repeated use. Furthermore, it is impossible for ultrasound machine manufacturers to determine presets that are optimal in all environments. These manufacturers rely on specialists, luminary input, anecdotal information obtained in interviews and experimental information gathered from ultrasound labs in order to determine the presets. Accordingly, there is a need for improved system and method for collecting information on adjusting and determining presets in ultrasound machines.

### SUMMARY

In view of the foregoing, the present invention contemplates a system and method that provide a better way for collecting information on adjusting and determining presets in ultrasound machines. The system and method according to various embodiments of the present invention involve automatic collection and analysis of changes to ultrasound scan parameters frequently used and optimized by the operator for various types of scan scenarios. Analysis of the automatically collected data (parameter changes) predicts presets more suitable to the clinical needs of the operator and the ultrasound machine operator is given the opportunity to accept modifications to the default presets or to prompt creation of new presets. Additionally, the preset changes may be made automatically. Moreover, the automatically collected information and any analysis associated with it are reported back to the manufacturing company and used for determining what default presets should be programmed in new machines. The report back is done in real time through wireless connection, network connection or both. The report back is alternatively done through use of a storage device or computer readable memory medium in which ultrasound scan settings are saved for later review. In other instances, the report back is put off until service or maintenance are done and, at that time, the report back is done either automatically or through a request made from the manufacturer's representative.

In other words, for the purpose of the invention as shown and broadly described herein, a system and method are provided for the automatic adjustment of presets. One embodiment of the system includes, among other things, an ultrasound machine having a front end with a transducer for sending and receiving acoustic signals, a processor, and a memory pre-programmed with a preset, wherein the preset is associated with an ultrasound exam and includes a parameter. Such system further includes an input device linked with the processor for adjusting the parameter during the ultrasound exam, and a storage device linked with the processor. The processor is responsive to the input device and is operative to modify a preset parameter. The processor is additionally operative, substantially coincident with the modifying, to communicate data of the parameter and any other parameters or data required or useful for the analysis to the storage device. The communication of the data allows for automatically establishing a historical preset record from which a pattern of frequent parameter adjustments is detectable and a new preset is identifiable.

In another embodiment, a system for providing automatically adjusted presets, includes an imaging device having a preset and an input device operative for adjustments of the preset and further having a transducer and a first processing computer for processing images obtained via the transducer. The system additionally has a storage device having storage space for a historical log of preset adjustments, and a second processing computer for accessing and analyzing the contents of the historical log. It is possible that a single processing computer is operative to carry out the functions of both the first and second processing computers.

Applying similar principles, a method for automatically adjusting presets in a system with an imaging device having a preset, includes the steps of gathering data on adjustments of the preset, wherein the adjustments are made to improve an image generated by the imaging device. This method includes also the step of creating a historical log of preset adjustments from the gathered data, and analyzing the historical log to determine if there is evidence suggesting automatic adjustment of the preset or creation of a new preset to thereby avoid repeat preset adjustments. Then, if the evidence suggests adjustment of the preset or creation of a new preset, the method further includes providing an alert to a user of the imaging device.

Another embodiment of the method involves obtaining a preset with a parameter that has a default value, the preset being obtained for an ultrasound scan. The method also includes obtaining a new value of the parameter which is different from its default value, the default value having been adjusted during the ultrasound scan. The method further includes converting received acoustic signals of the ultrasound scan into an image, the ultrasound resulting image being associated with the new value of the parameter. In addition, the method includes detecting an indicia of approval of the resulting image and, in response thereto, recording data associated with the preset and the new value of the parameter, the recorded data establishing a historical preset record from which a pattern of frequent parameter adjustments is detectable and a new preset is identifiable.

These and other features, aspects and advantages of the present invention will become better understood from the description herein, appended claims, and accompanying drawings as hereafter described. Moreover, while the description herein is made with respect to an ultrasound imaging device, the principles described are applicable to other medical imaging equipment which uses presets that can be modified by an operator.

### BRIEF DESCRIPTION OF THE DRAWINGS

The accompanying drawings which are incorporated in and constitute a part of this specification illustrate various aspects of the invention and together with the description, serve to explain its principles. Wherever appropriate, the same reference numbers are used throughout the drawings to refer to the same or like elements.

FIG. 1 illustrates one system in which the invention is embodied.

FIG. 2 is a flow diagram illustrating adjustment of parameters in an ultrasound scan procedure without the help of the automatically adjustable presets feature.

FIG. 3 is a flow diagram illustrating adjustment of parameters with the help of the automatically adjustable presets feature.

FIG. 4 is a flow diagram illustrating creation of a new preset without the help of the automatically adjustable presets feature.

FIG. 5 is a flow diagram illustrating creation of a new preset with the help of the automatically adjustable presets feature.

FIG. 6 (parts 1 & 2) is a flow diagram illustrating modification of a default preset and creation of a custom preset with the help of the automatically adjustable presets feature.

### DETAILED DESCRIPTION

The present invention is based, in part, on the observation that historical data on ultrasound scan presets is useful for determining default presets and for alerting an operator to frequently adjusted parameters. The present invention is based on the additional observation that a typical indicia of operator satisfaction with scan results is the invocation of printing or saving of the image representing the scan results, or the invocation of recording a measurement taken from a given image. This indicia is coincident with a state of the ultrasound machine in which all relevant scan parameters are considered to be set satisfactorily. The point at which this coincidence occurs can be defined as the point of approval. Then, because the salient issue in determining presets for ultrasound scans is the ability to obtain and make use of statistically more reliable data about scan parameters for various scan scenarios, the point of approval is suitably selected by the present invention as the point for recording the scan parameters. It is noted that although the indicia and point of approval defined above are preferred because they are easy to implement and produce relatively reliable results, other definitions may be possible. The present invention is based on yet another observation that gathering the historical data on scan presets helps identify patterns in ultrasound scan parameters adjustment. Identification of such patterns can be the basis for alerting an operator, for modifying default presets, and for creating custom presets.

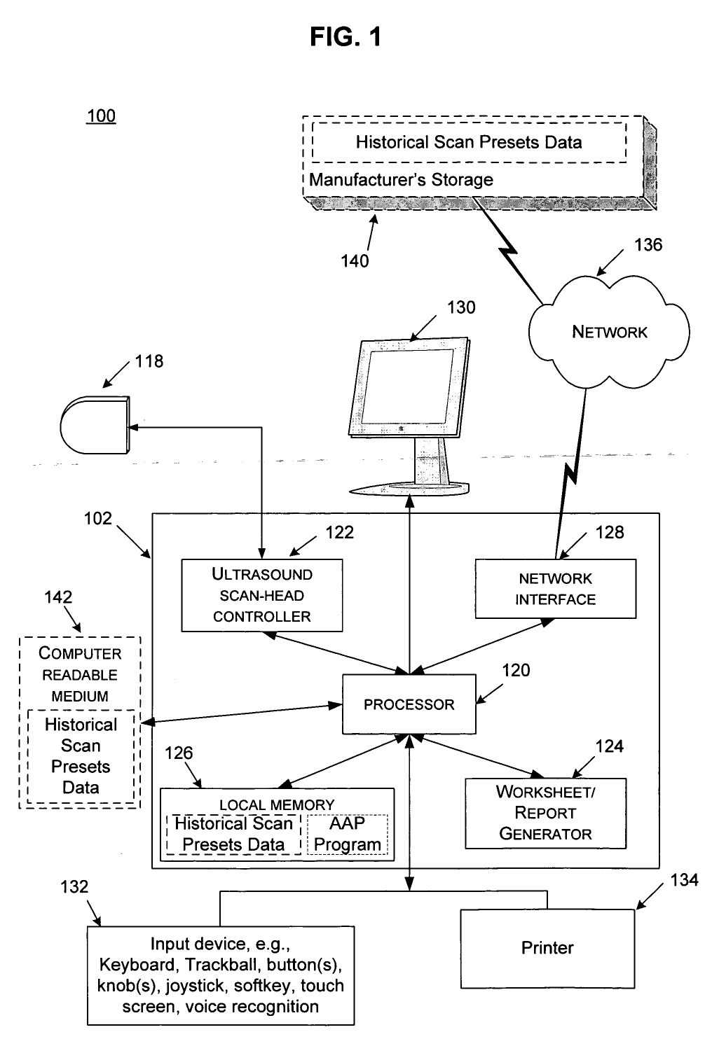

To that end, the system with the ultrasound machine as shown in FIG. 1 is deployed with an automatically adjustable parameters (AAP) feature. Such system 100 is set up to automatically collect data about scan parameters used by the ultrasound machine operator and find parameters that are more frequently adjusted. The ultrasound machine 102 includes components, such processor 120, memory 126 and input devices 132, for loading presets, adjusting parameters, analyzing results and more. Although for simplicity the system is illustrated with a single processor 120 ultrasound machines often include more than one processor. Typically, one processor handles user interface functions and another processor handles image data processing. In a system that embodies the present invention there is at least one processor, in the ultrasound machine or otherwise, for executing a program that implements the AAP feature (we refer to this program as the AAP software). The AAP software is stored at the local memory 126, in hard drive or otherwise. The AAP software is configured to perform the functions of automatic data collection, analysis, operator alert and more, as later described.

The input device 132 includes one or any combination of a trackball, a mouse, a joystick, a set of one or more hard keys, soft keys and knobs, a touch screen, a voice recognition device, etc. The ultrasound machine further includes output devices, such as a monitor 130 and a printer 134, for presenting the scan image generated via the processor 120. Often times, the printer 134 is a high-end, photo quality printer. The ultrasound scan-head 118 and scan-head controller 122 are part of the, so called, front-end of the ultrasound machine which also includes a beam-former and an A/D converter (not shown). As can be seen, the transducer is controlled indirectly by the processor and, typically, real-time control of the transducer is performed by a dedicated hardware such as an FPGA (field programmable gate array, not shown). The worksheet/report generator 124 records conclusions derived from the ultrasound scan in report format.

The system 100 is set up to gather historical data on scan presets and the historical presets log is saved either at the local storage 126 or on a separate storage device 142. The aforementioned AAP software is configured to automatically collect the data for the historical log, as well as to analyze it, create alerts to the operator and automatically adjust the presets. The system 100 may perform such functions autonomously or it may report back to the manufacturer. For reporting back to the manufacturer, the system 100 reports its findings directly or indirectly, in real time or asynchronously. In one instance, the manufacturer obtains the automatically collected data via a network (e.g., the Internet) 136. In another instance, the manufacturer gains access to the automatically collect data by retrieving the storage device 142 from the ultrasound machine 102 or by reading its content without removing it. A portable computer readable medium is an example of such storage device.

When a system deployed with the automatically adjustable presets feature, reports the findings, including the frequently adjusted parameters, to the ultrasound machine manufacturer, this information is used for determining improved default presets. Indeed, because of the automatically adjusted presets feature, each new generation of systems is preconfigured by the manufacturer with improved default presets based on the data obtained with a previous generation of systems. Thus, each system is deployed with functionality that includes this feature as further described below.

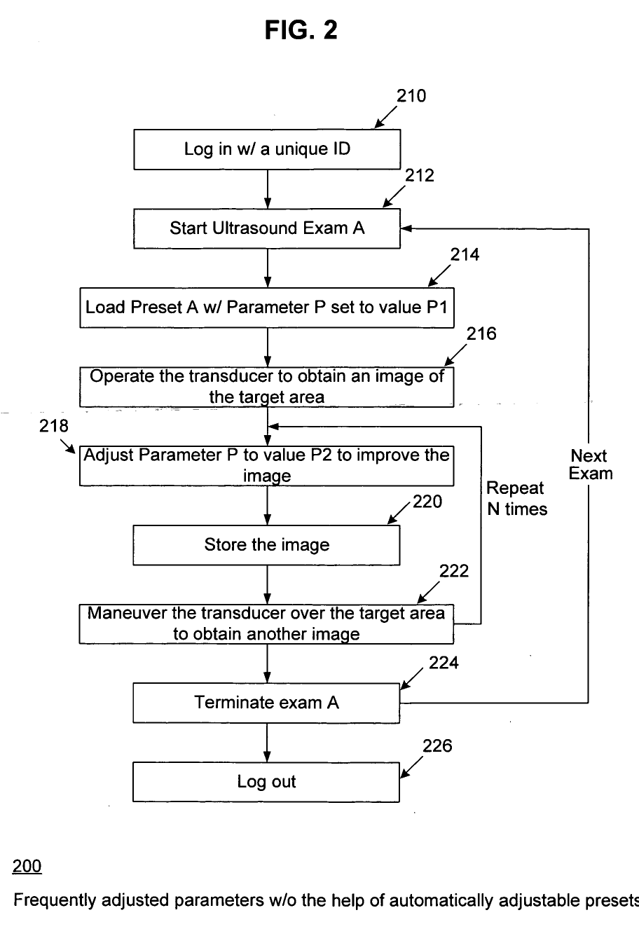

As a point of reference, FIG. 2 is a flow diagram illustrating adjustment of parameters in such a system during an ultrasound scan procedure without the help of the automatically adjustable presets feature. In this scenario, the ultrasound machine operator (referred to simply as “the user”) logs in with a unique identification 210 and starts the ultrasound exam 212. This ultrasound exam is hypothetically called Exam A. For this exam, the manufacturer may already have a preset suitable and automatically set for it or the user may select a suitable preset. Whichever the case may be, the ready or selected preset loaded by the machine (collectively referred to as the selected preset for simplicity) is hypothetically called Preset A. The selected preset includes a set of default scan and display parameters appropriate for the given ultrasound exam. Two of these parameters are hypothetically called Parameter P and Parameter S, respectively. Parameter P has a default value of P1 and Parameter S has a default value of S1.

Because different types of ultrasound exams require different settings, their respective presets are different, although they may share some of the same parameter settings. Here are some examples of presets. In one example, the suitable preset for B-Mode imaging covers a set of parameters including dynamic range, transmit power, frequency, scale map and color map (gray scale map and possibly tint). For color Doppler (CD) imaging the preset covers a set of parameters that include baseline, color map, dynamic range, filter closeness, transmit power and sharpness. For spectral imaging modes, continuous wave (CW) and pulse wave (PW) imaging modes, the preset covers a set of parameters that include frequency, transmit power, Doppler update interval, dynamic range, Doppler strip size, pulse wave steer angle, sharpness, spectral color map and gray scale map. For M-Mode imaging the preset includes parameters such as color map, color on, dynamic range, sharpness and transmit power. User interface preferences cover a set of parameters that include annotation level, auto freeze on store duration, default playback speed, number of stages, playback mode, maximum frame rate, abdomen marker, kidney marker, liver marker, monitor brightness and monitor contrast. Of course, the forgoing enumeration is merely illustrative and not intending to be an exhaustive listing of all parameters and presets.

Returning to FIG. 2, after Preset A is loaded, the user operates the transducer (within scan head 118) to obtain a scan image of the target area 216. To improve the image, the user adjusts one of more parameters 218. In this illustration the user adjusts parameter P from its default value of P1 to value P2 in order to achieve satisfactory representation of the scan results. Then, the user prompts the system to store the scan image 220. The user obtains additional images by maneuvering the transducer over the target area 222. Each time, the user stores the image 220. The number, N, of images obtained is determined by the user, and this determination is a matter of judgment exercised by the user for the particular exam scenario. In general, the number N is greater than one in order to allow sufficient accumulation of data from sampling of the target area in different angles. At the end of this exam 224, the user logs out 226.

The process just described does not use the automatically adjusted presets feature and, as a result, once Exam A is terminated there is no record of the settings used for this exam. If the user would want to recall the same exam settings the user would have to commit the settings to memory or, alternatively, disrupt the exam in order to save the settings using the preset store functionality via a ‘preset’ page.

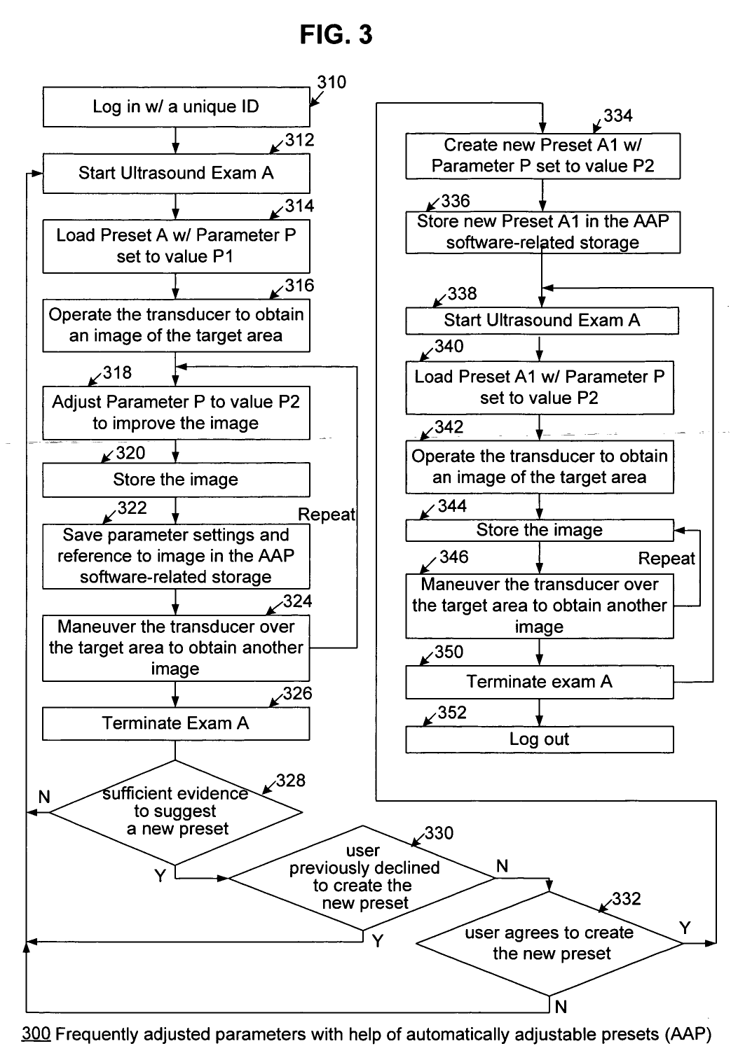

The better alternative, of course, is to use the automatically adjustable presets feature in the scan process. FIG. 3 is a flow diagram illustrating adjustment of parameters with the help of the automatically adjustable presets feature.

As shown, the user selects Preset A for the ultrasound exam, Exam A (312, 314). Once again the user adjusts Parameter P to value P2 (step 318). Actually, the adjustment of Parameter P could happen for each new image, and, although this adjustment is typically made by the user in order to improve the image other reasons may dictate it. For example, clinic or hospital procedures may dictate certain settings including Parameter P at value P2. Either way, the value P2 cannot be recalled because it was not previously recorded. This time, however, in addition to storing the image 320 the parameter settings (including P2) are saved in the storage device (142, FIG. 1) or even in the local memory if the manufacturer can gain access to them 322. In addition, or, alternatively, the parameter settings are reported to the manufacturer directly and stored in the manufacturer's storage. The reason for saving the parameter settings is twofold; the first reason being to allow recall of parameter settings and the second reason being to allow further analysis of the parameter settings (including establishing a pattern of frequently adjusted parameters). The current parameter change may be passed on to a concurrently running application program for analysis. The AAP program is configured to use the current parameter adjustment, system state, and historical log for analyzing the frequency of this parameter change and, if warranted, to suggest or make a preset change.

As before, the user maneuvers the transducer to obtain one or more images of the desired region. This time, however, each time an indicia of approval is detected, in this case the image is saved, the system saves reference data, such as exam type, along with the parameter settings 322. When the exam is about to be or has already been terminated, the system decides if there is sufficient evidence to suggest a new preset 328. Whether or not there is sufficient evidence depends in large part on the type of ultrasound exam and to what extent the same adjustments have been repeated a number of times, say three (3) times. Sufficient evidence does not necessarily mean that there is a need for lots of data; there can be less data with plenty of evidence. Assuming that there is sufficient evidence to suggest a new preset, and the user has not previously declined to create the new preset 330, the user can agree to create the new preset by responding to a prompt 332.

Incidentally, the operation of the automatically adjustable presets feature is transparent to the user and does not interfere with the exam procedure. The user is not aware for example that system saves the parameter settings or sends them directly to the manufacturer for further analysis, although the system may associate the parameter data with the user based on the user's unique log-in identification (user ID) and even the specific system and transducer being used. One difference the user notices is the prompt or dialog asking the user to agree to the new preset. If the user agrees, the system automatically creates the new preset, calling it Preset A1, and saves it in a designated storage 334, 336. Then, the user can start a new exam like Exam A and for this exam the user selects Preset A1 which is loaded with parameter P at value P2 (steps 338, 340). From the user's vantage point, the recurring procedure is similar except that the user is not required to adjust Parameter P in order to obtain a better image or achieve compliance with procedures.

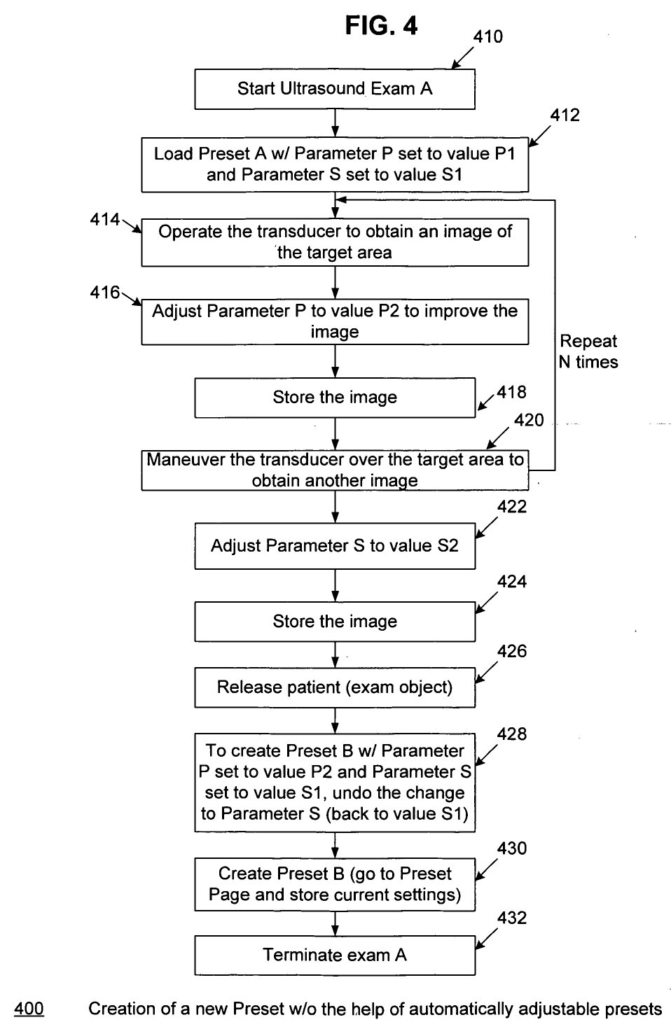

In the example just described, the new preset is presumed created automatically. However, as a point of reference, FIG. 4 is a flow diagram illustrating creation of a new preset without the help of the automatically adjustable presets feature.

Once again, the user starts the hypothetical Exam A and selects Preset A for it 410, 412. Preset A is loaded with a set of parameters, Parameter P with default value of P1 and Parameter S with default value of S1. The user operates the transducer to obtain and image and improves the image by adjusting Parameter P to P2 (steps 414, 416). Further maneuvering of the transducer produces additional images 420, and each time an image is obtained it is stored 418. For further improvement, or in accordance with operation procedures, the user also adjusts Parameter S to value S2 (step 422). The image obtained as a result is likewise stored 424, and the patient is released 426.

In this case, the user notices the frequent adjustments of Parameter P to value P2 and decides to create a new preset, calling it Preset B (step 428). However, to create Preset B with Parameter P set to value P2, the user must first reconstruct the-change to Parameter P from P1 to P2. That is, the user must first go to the preset definition page to adjust Parameter P to P2; and this time leave Parameter S untouched. Moreover, to create Preset B, the user goes to the ‘preset’ page and uses the prompts in this page to store the current settings. This procedure is manual and requires that the user commit to memory the S1 setting.

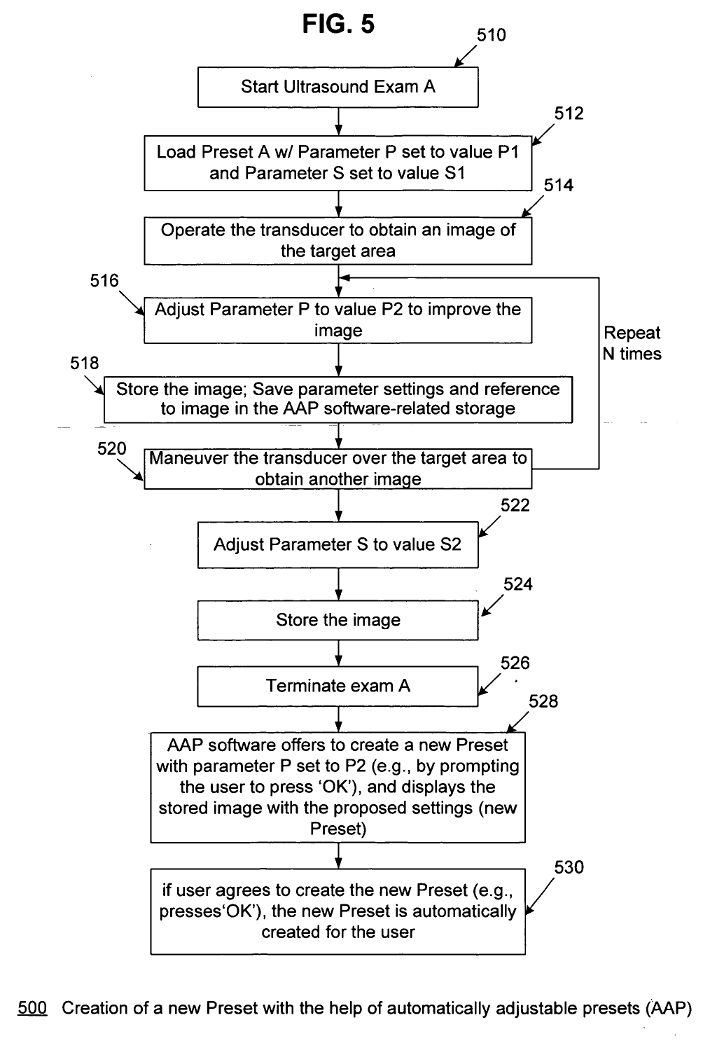

Therefore, the better approach employs the automatically adjustable presets feature. FIG. 5 is a flow diagram illustrating creation of a new preset with the help of the automatically adjustable presets feature.

As shown, the exam proceeds as in previous examples in which Parameter P is adjusted to value P2 and a series of scan images is obtained and stored (steps 510, 512, 514, 516, 518, 520). Parameter S is then adjusted to value S2 and another image is obtained and stored before Exam A is terminated (steps 522, 524, 526). In the background, the system keeps track of the images and parameter settings employed by the user throughout the ultrasound exam. Keeping track of this data involves saving the data in a storage device, a local memory, a remote location via a communications link, or any combination of these facilities. For instance, at each point of approval when indicia of approval is indicated, the associated parameter settings are also stored by the system in a storage location. The parameter settings and associated information (which may include exam information, patient information, user identification, and equipment information) are logged (stored locally, or remotely). This data may be reported to the equipment manufacturer via the network if the system is set up for such communication or they are stored in a storage device that can be delivered to or at least accessed by the equipment manufacturer.

Whichever way the system keeps track of the data, an analysis program within the system detects patterns of frequently adjusted parameters. Upon finding such a pattern, the system initiates a dialog with the user in which it offers to the user to create a new Preset 528. The new (custom) preset will include the frequently adjusted parameter at its preferred value, namely, Parameter P set to value P2. At a push of a button, the user can agree to create the new preset, say by pressing ‘OK’ button 530. In an alternative embodiment, the system updates the parameter without user interaction. In yet another embodiment, the system reports only the preferred setting for the environment back to the manufacturer.

Notably, the user need not track or commit to memory any presets or frequently adjusted parameters. The user need not detect any pattern or determine that it justifies creating a custom preset. The system does it for the user, automatically and transparently.

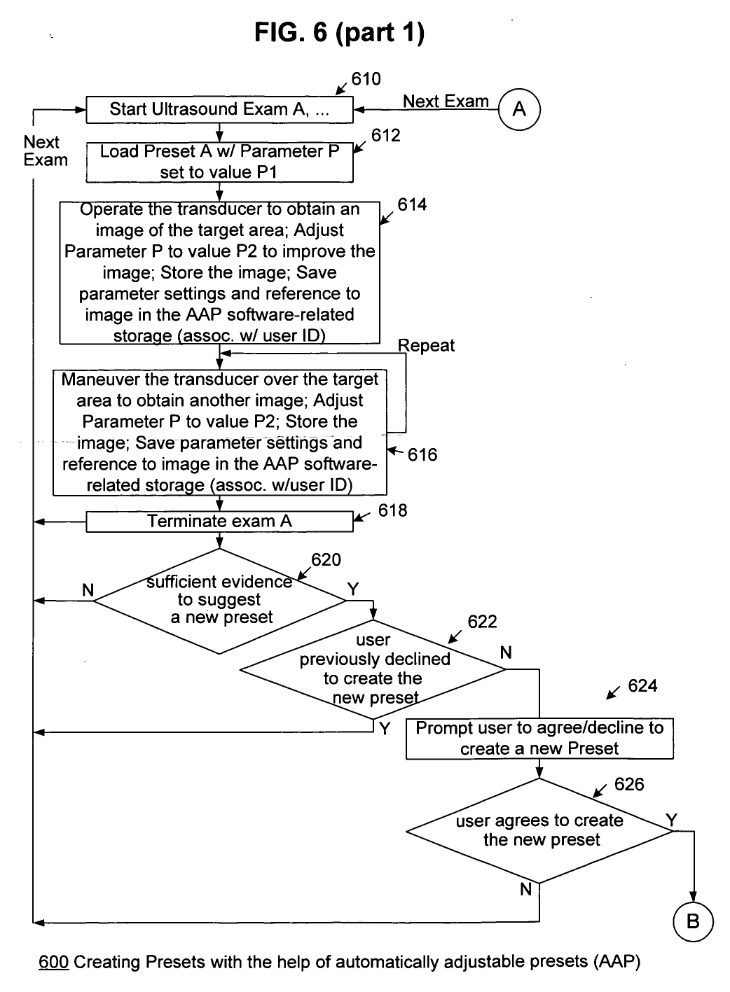

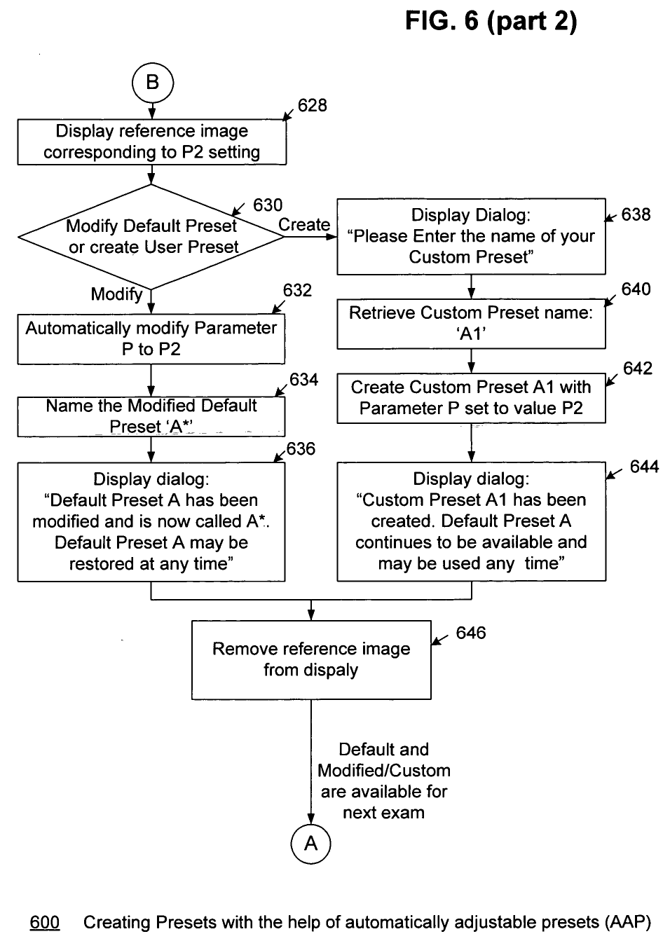

Indeed, in using a system deployed with the automatically adjustable presets feature, the user can choose to create a custom preset, create a modified default preset, or restore a default preset. FIG. 6 (parts 1 & 2) is a flow diagram illustrating modification of a default preset and creation of a custom preset with the help of the automatically adjustable presets feature.

As shown in FIG. 6 part 1, assuming again that the user starts Exam A, the user prompts the system to load Preset A with Parameter P set to value P1 (steps 610, 612). The user continues the exam by maneuvering the transducer, adjusting Parameter P to value P2 and providing an indicia of approval by prompting the system to store the image 616. This, in turn, prompts the system to log the parameter settings in a manner as discussed before. Upon terminating the exam 618, the system analyzes the parameter settings data it recorded in order to determine if there is sufficient evidence to suggest a new preset 620. The determination is based for instance on the number of times Parameter P has been adjusted in previous exams of a particular type conducted by the particular user within a day. Assuming that there is data showing that the user will be more likely to adjust Parameter P to value P2 than leave it in its default state, this is considered sufficient to suggest a new preset adjustment. Thus, the system then determines whether the user has previously declined to create the new preset or modify the default preset 622. If the user has not declined, the system introduces a query or dialog box asking the user to respond 624. If the user has previously declined or declines in the present instance, the system does not urge the user to continue and the user is free to start a new exam (with the pre-existing presets). Otherwise, the system proceeds with the process of creating the custom or modified preset.

As shown in FIG. 6 part 2, the system queries the user as to whether the user wishes to modify the default preset or create a custom preset 630 (in order to avoid the need for repeated adjustments of Parameter P to value P2). If the user chooses to create a custom preset, the system introduces a dialog prompting the user to enter a name for the custom preset 638. The hypothetical custom preset name is Preset A1. After obtaining from the user the name of the custom preset 640, the system automatically creates Preset A1 without further intervention by the user 642. However, the system displays a message to the user indicating that the default preset is still available and can be recalled at any time 644.

If the user chooses to modify the default preset instead of creating a custom preset 630, the system automatically modifies Parameter P to value P2 and saves it in a modified default preset called A*, without further user intervention 636. The system displays a message to the user indicating that the default preset can be restored at any time 636. At the close of this process the system removes the reference image from the display 646. Then the user is free to start a new exam (step 610) with the modified, custom or default presets. For security purposes, the system may limit modification of system presets to a limited set of users.

The automatically adjustable parameters feature can be particularly useful in connection with an intuitive user interface where parameters are controlled by selecting points in a parameter space rather than changing one parameter at a time. The intuitive user interface concept as briefly described below is implemented in ultrasound imaging to improve the interplay between various parameter controls.

Normally, adjusting a parameter to optimize the system for a particular image quality aspect would unavoidably affect one or more of the remaining aspects. Indeed, every preset affects these aspects; some effects are positive and other effects are negative. Thus an intuitive user interface is preferably implemented in ultrasound imaging to improve this interplay. Conceptually, an intuitive user interface integrates some or all of the image quality parameter controls into a 4-pole user control, with one pole for each image quality aspect (detail resolution, contrast, temporal resolution and sensitivity/penetration); and it adopts the 4-pole user control for the presets. One way to implement the 4-pole user interface is by providing a device that allows the user to smoothly maneuver between points on the surface of a triangle (e.g., trackball or joystick). The triangle is defined by three corners corresponding to three of the four parameters. The center of the triangle represents the fourth parameter. The finite number of grid points in the triangle domain provide a relative weighing (preference) of parameters. The center of the triangle corresponds to a default setting where the fourth parameter is emphasized and the emphasis of the other three parameters is equal. Operating points along any of the triangle edges indicate de-emphasizing of the opposite corner's parameter.

Hence, with the intuitive user interface, as the user moves from one point to another in the parameter space (to shift emphasis between parameters), the user may venture into an area not previously covered by an existing preset, although this area may include parameter settings that are useful for the particular imaging scenario. Then, with automatically adjustable presets, the system collects data about the user's maneuvering over the parameters domain and data as to where the user stops more frequently and saves the images. This allows the system to identify candidates for new presets. In other words, the combination of automatically adjustable presets and intuitive user interface further improves the user experience and more readily achieves the advantages mentioned above.

In sum, the present invention is implemented in a system and method for providing automatically adjustable presets as illustrated in the drawings and described above. Although the present invention has been described in considerable detail with reference to certain preferred embodiments thereof, other embodiments are possible. Therefore, the spirit and scope of the appended claims should not be limited to the description of the embodiments contained herein.

What is claimed is:

1. A system for adjusting presets, comprising:
an imaging device having a preset and an input device operative for adjustments of the preset and further having a transducer and a first processing computer for processing images obtained via the transducer;
a storage device having storage space for a historical log of preset adjustments; and
a second processing computer for accessing and analyzing the contents of the historical log.

2. A system as in claim 1, wherein a single processing computer is operative to carry out the functions of both the first and second processing computers.

3. A system as in claim 1, wherein the second processing computer is operative to automatically adjusts the preset.

4. A system as in claim 1, wherein the second processing computer is operative to recommend to a user an adjustment of the preset.

5. A system as in claim 1, wherein the second processing computer is operative to recommend to a manufacturer an adjustment of the preset.

6. A system as in claim 1, wherein the transducer is maneuverable to obtain scan results from different positions, and wherein the first processing computer is operative to produce an image for each of the scan results and communicate data of the parameter associated with each image.

7. A system as in claim 1, wherein the preset is configured as a default preset with a parameter having a default value adjustable to a new value by the input device.

8. A system as in claim 7, further comprising a display for user interface prompts including queries about user's approval to create a modified or a custom preset which includes the new value of the parameter.

9. A system as in claim 1, wherein the storage device is local to, removable from, or remote to the first or second processing devices.

10. A system as in claim 1, further comprising a network interface and a network for communication to a manufacturer's computer.

11. A system as in claim 1, wherein the imaging device is an ultrasound machine.

12. A method for adjusting presets in a system with an imaging device having a preset, comprising:
gathering data on adjustments of the preset, wherein the adjustments are made to improve an image generated by the imaging device;
creating a historical log of preset adjustments from the gathered data;
analyzing the historical log to determine if there is evidence suggesting automatic adjustment of the preset or creation of a new preset to thereby avoid repeat preset adjustments.

13. A method as in claim 13, wherein if the evidence suggests adjustment of the preset or creation of a new preset, the method further comprises providing an alert to a user of the imaging device.

14. A method as in claim 12, wherein the historical log and any suggestion are reported to a manufacturer of the imaging device.

15. A method as in claim 12, wherein the imaging device is an ultrasound machine.

16. A method of adjustable presets, comprising:
obtaining a preset with a parameter that has a default value, the preset being obtained for a scan;
obtaining a new value of the parameter which is different from its default value, the default value having been adjusted during the scan;
converting results of the scan into an image, the scan results and image being associate with the new value of the parameter; and
detecting an indicia of approval of the scan results and, in response thereto, recording data associated with the preset and the new value of the parameter, the recorded data establishing a historical preset record from which a pattern of frequent parameter adjustments is detectable and a new preset is identifiable.

17. A method as in claim 16, further comprising associating the pattern of frequent parameter adjustments with the parameter and producing an alert that a frequent adjustment pattern has been identified and suggests a new preset.

18. A method as in claim 16, wherein the data recording includes saving the data to a storage device which is local, portable or remote.

19. A method as in claim 16, further comprising introducing a dialog when detecting a pattern of frequent parameter adjustments, wherein the dialog includes a query as to whether a modified preset or a custom preset should be created.

20. A method as in claim 16, further comprising creating a modified preset or a custom preset with the parameter set to the new value, and displaying a message indicting that the preset with the default value is restorable.

21. A method as in claim 16, further comprising analyzing the historical preset record to determine which new presets are suggested thereby as replacement for the preset or as additional default presets.

22. A method as in claim 16, wherein the scan is an ultrasound scan.

23. A system for adjusting presets having an imaging device with a preset, comprising:
means for gathering data on adjustments of the preset, wherein the adjustments are made to improve an image generated by the imaging device;
means for creating a historical log of preset adjustments from the gathered data;
means for analyzing the historical log to determine if there is evidence suggesting automatic adjustment of the preset or creation of a new preset to thereby avoid repeat preset adjustments.

24. A system as in claim 23, wherein if the evidence suggests adjustment of the preset or creation of a new preset, the system further comprises means for providing an alert to a user of the imaging device.

25. A system as in claim 23, wherein the historical log and any suggestion are reported to a manufacturer of the imaging device.

26. A system as in claim 23, wherein the imaging device is an ultrasound machine.
